(19) United States
(12) Patent Application Publication
(10) Pub. No.: US 2016/0054410 A1
(43) Pub. Date: Feb. 25, 2016

(54) SYSTEM AND METHOD FOR LOCATING AND QUANTIFYING A BIOMARKER FOR NEUROLOGICAL DISEASE

(71) Applicant: GENERAL ELECTRIC COMPANY, SCHENECTADY, NY (US)

(72) Inventors: John Frederick Schenck, Voorheesville, NY (US); Ek Tsoon Tan, Mechanicville, NY (US); Dominic Michael Graziani, Loudonville, NY (US); Suresh Emmanuel Devadoss Joel, Bangalore (IN)

(21) Appl. No.: 14/826,468

(22) Filed: Aug. 14, 2015

Related U.S. Application Data
(60) Provisional application No. 62/039,080, filed on Aug. 19, 2014.

(51) Int. Cl. G01R 33/48; G01R 33/50; A61B 5/055

(52) U.S. Cl. CPC G01R 33/4806; A61B 5/055; G01R 33/50

(57) ABSTRACT

In embodiments of the invention, the habenulae have been identified and localized in normal volunteers. Aspects of the invention determine the location, volume and magnetic susceptibility of the habenulae. Furthermore, diagnosing and monitoring patient disorders are enabled using the herein disclosed methodologies and techniques.

### PRIORITY CLAIM

The present application claims priority under 35 U.S.C. §119 as a nonprovisional application of Provisional Application Ser. No. 62/039,080, titled System and Method for Locating and Quantifying a Biomarker for Neurological Disease, filed Aug. 19, 2014, the content of which is hereby incorporated by reference into this application.

### FIELD

The subject matter disclosed herein relates to magnetic resonance imaging (MRI), particularly as it relates to an MRI methodology as to quantitative susceptibility reconstruction for locating and characterizing the human habenulae as biomarkers for neurological and psychiatric diseases.

### BACKGROUND

Treatment of many neurologic and psychiatric diseases has been severely hampered by the lack of objective measures to determine the presence, progression and response to therapy of specific disorders. This leads to ambiguity and uncertainty in the diagnosis and treatment of these disorders and the need to rely on subjective measures in determining whether a particular chosen treatment method is effective or should be discontinued or modified.

Magnetic Resonance Imaging (MRI) is capable of producing in vivo images containing information such as physical properties (T1, T2), tissue structure, motional properties (velocity, diffusion), temperature, and mechanical properties (stiffness, etc.). MRI is also able to provide information regarding the electrical and magnetic properties of tissue. The motivation to produce and analyze images of magnetic properties has both clinical and research interests.

In view of magnetic property imaging, the susceptibility of tissue has been a topic of recent research. Specifically, phase images have been shown to have well defined clinical applications and usage. Further, the susceptibility values can be quantified using quantitative susceptibility mapping (QSM) approaches. These quantitative approaches require advanced processing methods to effectively quantify the susceptibility as a function of position within the body.

A static magnetic field is used by magnetic resonance imaging (MRI) scanners to align the nuclear spins of atoms as part of the procedure for producing internal images of a patient's body. A major difficulty is visualizing and identifying known and unknown regions or attributes of the brain when these structures are very small in size or lack conspicuous contrast in MRI images.

Several techniques for demonstrating MRI-based measures of depression have been proposed but none to date has been completely validated and clinically accepted. The term habenula (plural, habenulae) refers to two small and visually inconspicuous cell masses located deep in the brain on either side of the third ventricle. They are interconnected across the third ventricle by a small bridge of nerve fibers, the habenular commissure, and are in close proximity, but not connected by nerve fibers, to the pineal gland. A bundle of nerves, the stria medularis, brings signals from various important brain regions to the habenulae. Another fiber bundle, the fasciculus retroflexus, carries signals originating in the habenulae to important brainstem nuclei which control much of the brain's production of neurotransmitters such as serotonin and dopamine. Taken together the habenulae and their commissure along with the pineal gland and these two fiber tracts comprise the important brain region known as the epithalamus. The limbic system of the brain is believed to govern emotion, behavior, mood and other basic human functions. The habenulae are located at the crossroad of the limbic system with other basic brain systems. The habenulae are generally not seen or are very difficult to identify and characterize at high resolution using available imaging techniques such as PET, CT, diffusion-weighted or functional MRI. Thus, there is a need to render the habenulae more routinely conspicuous and to permit their quantification in terms of location and composition such as Fe and myelin content.

Despite substantial efforts, noninvasive objective biomarkers of psychiatric disorders (e.g. such as for major depressive disorder (MDD)) have not been developed or validated to date. A few other conventional MRI techniques have been utilized to visualize the habenulae but these tend to be of relatively low resolution (diffusion and functional MRI) or to require specialized and relatively unavailable MRI machinery, such as seven tesla MRI scanners. Thus, these alternative MRI methodologies provide incomplete results with limited utility in characterizing this small region. Other imaging techniques, computerized tomography (CT) and positron emission tomography (PET), in particular, have the potential for providing information on brain disorders; yet, these techniques lack the sensitivity (i.e. for CT) and spatial resolution (i.e. for PET) to provide clinically useful information on the habenulae.

A need exists to address the diagnosis and treatment of psychiatric disorders. MRI in combination with susceptibility imaging will provide the capabilities to address these needs such that a system and method will be provided to diagnose and monitor therapeutic responses. Advantageously, treatments will be made available and adjusted according to treatment plan. Further, the proposed invention will address diagnosis and treatment of depression as tied to the habenulae and will provide capabilities that distinguish neurological structures of the brain corresponding to particular disorders. Further, these developments will enable those skilled in the art to extend this methodology to the diagnosis and treatment of other neurological and psychiatric disorders.

### SUMMARY

The above and other drawbacks or deficiencies may be overcome or alleviated by development of a system as described as follows. In one embodiment of the invention, the habenulae has been identified and localized in normal volunteers. Aspects of the invention allow the precise location, volume, and magnetic susceptibility of the habenulae to be determined. Furthermore, diagnosing and monitoring patient disorders are capable using the herein disclosed methodologies and techniques.

Embodiments disclosed describe a method for diagnosing disease and monitoring therapeutic response, the method comprising the steps of: identifying at least one neuroanatomical structure in a computed magnetic resonance image, wherein said at least one neuroanatomical structure has at least one biomarker; generating a magnetic susceptibility map of the at least one neuroanatomical structure based on the computed magnetic resonance image; and quantifying the biomarker associated with the at least one neuroanatomical structure to determine a diagnosis.

The method further comprises a step of monitoring the biomarker over time to diagnose a neurological condition or monitor response to a treatment plan. In the step of monitoring the biomarker over time, the composition of brain matter or a therapeutic agent is quantified. The biomarker is monitored to determine a therapeutic response, wherein said at least one neurological structure is one or more habenula. In another aspect, a step of utilizing a therapy is targeted to the habenulae and monitoring the therapy. One neurological condition diagnosed may be depression. In such diagnosis, the habenulae are associated with depression. The treatment plan may be adjusted based on one or more quantitative characteristics of the biomarker.

During the step of generating a magnetic susceptibility map, iron concentration is measured in brain tissue. Another step includes repeating the steps of generating the magnetic susceptibility map and quantifying the biomarker. The repetition occurs within hours or days as opposed to earlier methods of repetition over months or years. The shorter duration periods for repeating the quantification provides for rapid diagnoses, including diagnosing disease, planning treatment, modifying treatment, and studying diseases in regions of a mammalian brain.

Further, the method comprises a step of seeding a fiber tract map in a diffusion dataset to identify nerve bundles attached to at least one habenula. During the step of seeding, a seed is provided to be utilized with a diffusion tensor image (DTI) to specify the nerve bundles.

Specifically, quantitative measurements of the status of the habenulae provide guidance to medical practitioners for management of common disorders such as depression and bipolar disorder. Further, these developments can be extended in methodologies as to the diagnosis and treatment of other disorders of thought, mood and movement such as schizophrenia, bipolar disorder and Parkinson's disease.

### BRIEF DESCRIPTION OF THE DRAWINGS

FIG. 1 depicts the designation of the habenulae in a 3T QSM image at low magnification in a human brain.

FIG. 2 depicts the identification of the habenulae among other brain structures in a 3 T QSM image of a human brain.

FIG. 3 illustrates the brain structures at high magnification in a 3 T QSM image.

FIG. 4 illustrates susceptibility maps of the habenulae acquired every 12 hrs over the course of a 24 hour period. Susceptibility is displayed in parts per million (ppm) relative to the average susceptibility of cerebral spinal fluid (CSF) in the third ventricle.

FIG. 5 represents an embodiment that demonstrates temporal variation.

FIG. 6 depicts a T2 weighted image and an R2* map, as compared to the QSM image.

FIG. 7 illustrates an embodiment utilizing habenular tracts.

### DETAILED DESCRIPTION

Various embodiments will be described more fully hereinafter with reference to the accompanying drawings. Such embodiments should not be construed as limiting. For example, one or more aspects can be utilized in other embodiments and even other types of systems and methodologies. Referring to the drawings in general, it will be understood that the illustrations are for the purpose of describing particular embodiments and are not intended to be limiting.

A technique now being developed for the management of severe depression and other brain disorders is the use of deep brain stimulation (DBS), or neuromodulation, using electrodes inserted precisely into specific brain regions. Consequently, there is also a need for imaging capability to identify and localize small and inconspicuous structures such as the habenulae, either as targets for DBS or as landmarks that permit the more precise localization and targeting of neighboring brain structures.

For neurologic and psychiatric disorders, interest is focused on the susceptibility as a function of position within the brain. Characterization of brain iron (Fe) and myelin content has been achieved by measuring changes in transverse relaxation times (T2 and T2*) in magnitude MR images, particularly at higher magnetic field strengths. They have also been quantified in high-pass filtered phase images in the technique of susceptibility-modulated imaging. These techniques, however, have not been sufficiently sensitive to identify and characterize very small but iron-containing regions such as the small habenulae structures; let alone having any capability to provide clear-cut characterizations of habenulae location or properties. That is, because deconvolution with the a dipole kernel to transform the phase image into a susceptibility map eliminates the blurring due to blooming of Fe-rich regions in the phase and magnitude images, it yields a sharper (more conspicuous) representation of small structures such as the habenula.

Embodiments of the invention herein disclosed are related to imaging techniques to provide capabilities to enhance the effectiveness and safety of deep brain stimulation (DBS): (i) insertion of electrodes directly into the habenulae (or nearby structures localized by their proximity to the habenula) for treatment of certain diseases aided by the imaging techniques; and (ii) the designation of the precise location of the habenulae to provide landmarks permitting an improved localization of neighboring structures that have been selected for DBS.

The invention disclosed herein is the application of MRI Quantitative Susceptibility Mapping (QSM) to the location and characterization of the habenulae of the brain for the diagnosis, monitoring, stratification and treatment of neurological and psychiatric diseases. The human habenulae are two very small (a few cubic millimeters each) structures deep in the brain. Despite their small size and inconspicuous appearance, the habenulae are believed to be major determinants of human activities such decision making, response to stress and sleep as well as health issues such as depression and drug addiction. Because of their relation to the limbic system, the habenulae are being proposed to play a role in human thought and emotion. Embodiments of the invention utilize a recently developed MRI technique, Quantitative Susceptibility Mapping (QSM), to localize and characterize the habenulae. It has been realized that these small structures can have a strong bearing on human mood and thought disorders, for example, in the very common disorder MDD (major depressive disorder), schizophrenia and so on. As such, embodiments of the invention address a major unmet medical need for objective biomarker measures of such disorders for purposes of diagnosis, monitoring of therapy, and stratification of disease severity.

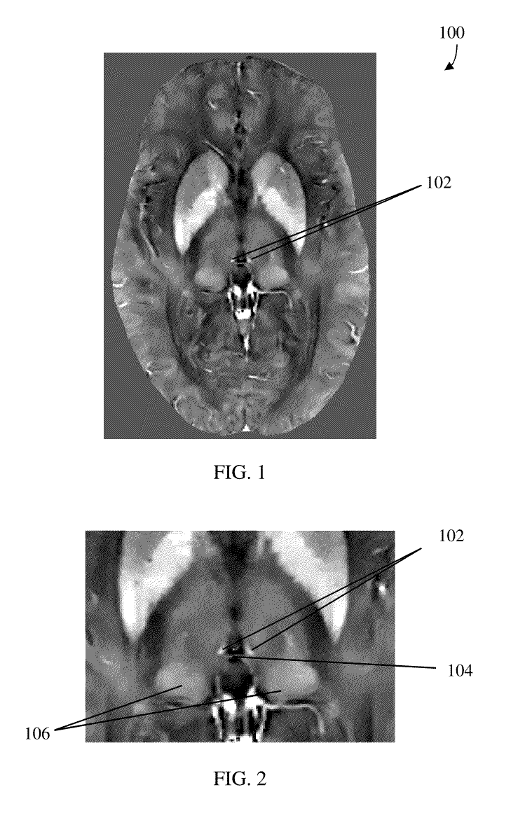

Various embodiments of the invention also provide improved precision for visualization and location of internal body structures, such as in the brain, for exemplary purposes and not limitation. As shown in FIG. 1, the human brain 100 is imaged using 3 tesla QSM at low magnification. Each habenulae 102 are identified by this imaging technique. The improved visualization and localization of these brain structures permits the accurate placement of neuromodulating electrodes for use in deep brain stimulation therapy of severe depression and related disorders. By use of the recently developed technique of QSM, the habenulae can be detected, located and characterized in a conspicuous fashion not provided by alternative imaging methodologies. The image contrast that demonstrates the location, volume and status of the habenulae is attributed to the presence of iron oxides and the presence of myelin sheaths surrounding nerve cells which differentiate these regions from the surrounding tissues. These contrast mechanisms are present in other brain regions but have not previously been demonstrated so evidently in the human habenulae.

FIG. 2 depicts the identification of the habenulae 102 and other brain structures in a 3 T QSM image of a human brain at medium magnification. The habenular commissure 104 is identified as well as pulvinar 106.

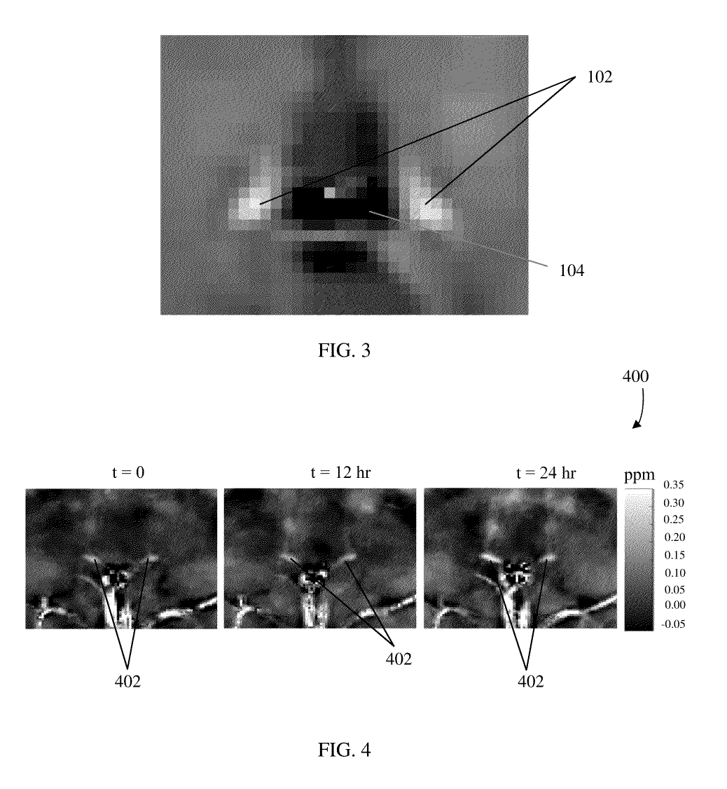

FIG. 3 illustrates the habenulae 102 and habenular commissure 104 at high magnification in a 3 T QSM image. The intensity of the pixels correlate to the iron quantification in the structure.

In one embodiment of the present invention, the technique involves placing the patient being studied within an MRI scanner operating at 3 tesla (3 T). Both higher and lower fields may be used, as desired, to achieve advantages of cost, accessibility, contrast and/or resolution. In one approach to this study, QSM images of the brain are acquired using axial slices with a slice thickness on the order of 1-3 mm. For a slice thickness of approximately 2 mm, the habenulae can be identified as very small regions of strong paramagnetism (relative to water). This is done by using as landmarks neighboring, but larger, brain structures that are prominent on QSM images. These structures include the third ventricle, the pulvinar of the thalamus, the internal capsule, the putamen, the globus pallidus and the caudate nucleus.

In one aspect, the habenulae are localized mainly to one or two adjacent axial slices containing the above landmark structures with possibly some presence in a slice immediately above or below these central slices. Once the habenulae have been identified, the image information can be used in a variety of ways to improve the management of the patient's disease. For exemplary purposes, the following studies have been coordinated: (i) Spatial coordinates of the habenulae are available from the image and can be used to direct the insertion of treatment probes with a greater degree of accuracy and safety than currently possible. (ii) The volume of the habenulae can be established by determining the number of voxels exhibiting increased paramagnetism. This has proved useful where studies of postmortem brains have indicated that the volume of the habenulae is decreased in patients with depression. (iii) Measurements of the magnetic susceptibility of the habenulae provide information on its iron oxide and myelin composition. By analogy with other brain disorders, this provides an objective measure of disease presence and progression. (iv) The results of the QSM imaging of the habenula can be combined with other imaging information such as MR diffusion-weighted imaging (DWI) to provide additional measures of disease.

Furthermore, the QSM image data is interpreted more readily in a quantitative fashion in terms of the direct iron and/or myelin content of the habenulae, as opposed to current methodologies. In comparison, other attempts to visualize the habenulae using MRI have utilized postmortem tissues (an obvious disadvantage) and/or magnitude images rather than QSM images. The magnitude images are based on the reduced transverse relaxation times (T2 or T2*) associated with the iron oxide content of the habenulae. FIG. 6 depicts T2 weighted images 601 and an R2* map 602, as compared to the QSM image 603. For reference purposes, R2 is a relaxation rate. It is the inverse of T2 which is the spin-spin relaxation time (i.e., dephasing due to field perturbations caused by the magnetic nuclei in the sample). R2* is the inverse of T2* which is relaxation caused by dephasing due to magnetic field perturbations from the environment, for example, from a magnetic source such as iron.

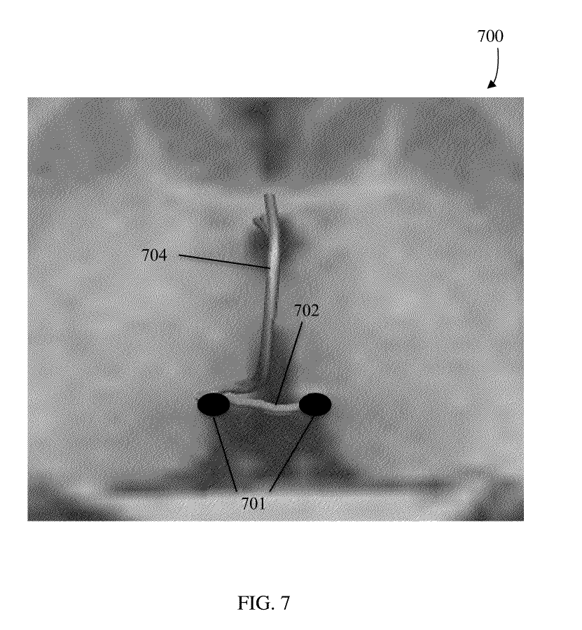

The contrast on magnitude images is much less distinct and difficult to interpret than that provided by the QSM, as shown. Using QSM, contrast of the habenulae is determined using 3 tesla scanners, much more widely available in clinical practice than are 7 tesla machines. The QSM technique can be combined with diffusion MRI to provide combined information on iron and myelin status of the habenulae which is not available from magnitude images alone. The QSM images can be used to seed a fiber tract map in a diffusion dataset to identify nerve bundles attached to the habenulae. FIG. 7 depicts an embodiment of a fiber tract map 700 using diffusion tractography obtained by seeding at the habenular nuclei 701; the habenular commissure 702 and the left stria medullaris 704 are identified as well.

In addition, the QSM images are acquired in a relatively short (approximately six minutes for the whole brain, or less than a minute for thin slab comprising the habenulae) gradient-recalled-acquisition (GRE) which is a standard MRI clinical sequence. Thus, the QSM study can be performed with a minimal or no increase in patient time within the scanner.

Embodiments encompass temporal variation as demonstrated by the images of FIG. 4. The susceptibility maps 400 of the habenulae 402 were acquired every 12 hrs over the course of a 24 hour period. The magnetic susceptibility (χ) is displayed in parts per million (ppm) relative to the average susceptibility of cerebral spinal fluid (CSF) in the third ventricle. The intensity of the habenulae changes over time, specifically the magnetic susceptibility, as suggestive of the iron concentration in the habenulae, the intensity increasing from t=0 to t=24 hours. In one embodiment, the variations in volume or magnetic susceptibility of the habenulae are determined over time through multiple scanning sessions. These sessions may take place over the course of hours, days, weeks, or years. The change in volume or magnetic susceptibility, as attributed to iron concentration, can thus be monitored over time in order to study the progression of a neurological condition, a response to treatment, or natural variations due to neurological state (mood, sleep, etc.).

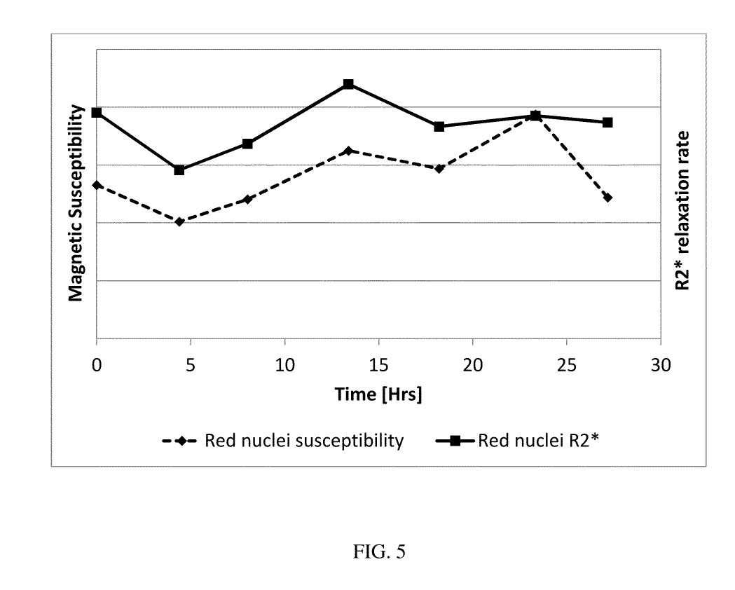

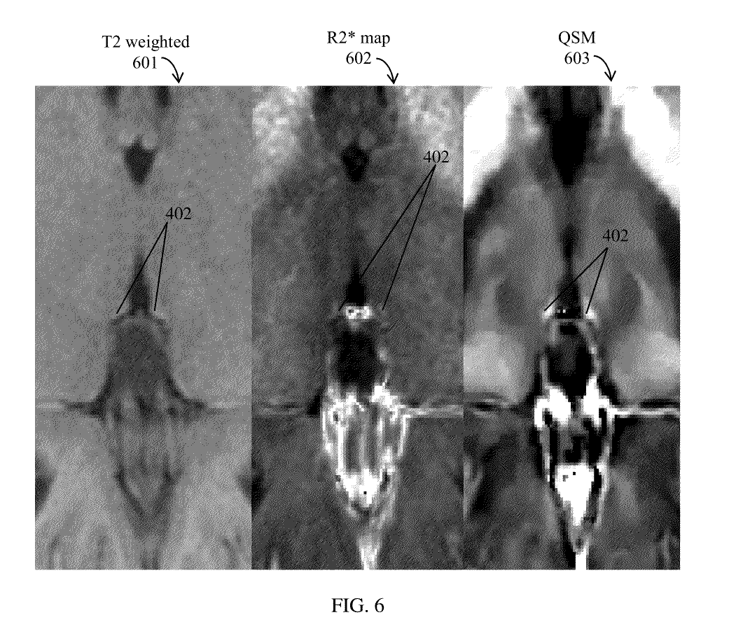

For exemplary purposes, and not limitation, FIG. 5 is utilized to demonstrate that over time variations in both magnetic susceptibility and the relaxation rate of the red nuclei are consistent, which infers that temporal variation is real, and thus characteristic of the variation seen in the habenulae as well. As depicted, the relaxation rate and the susceptibility change together. This provides evidence that the observed variation is physiological.

Various embodiments of the invention may encompass any number of designs, shapes, sizes, dimensions, as well as various image acquisition methodologies, timing and scanning sequences, as discussed above. As described herein for exemplary purpose, the biomarker is the habenula. Any iron-containing brain region, however, may be targeted, however, including the red nucleus, the substantia nigra, the globus pallidus, the putamen, the caudate nucleus, the hippocampus, the amygdala, the cortex and the nucleus basalis of Meynert, among others. In addition, Fe is characterized in the habenulae, but other materials such as the myelin and white matter may be characterized as well using QSM. While individual embodiments have been thus described, the individual embodiments of the MRI methodology and the identification of biomarkers may be integrated and combined for use in the characterization of disease and further diagnosis and treatment planning. While previous efforts have attempted to link MRI-detected changes in brain iron to diseases such as Parkinson's disease, these have assumed the observable changes over time periods of many months or years to become apparent. We have demonstrated these changes can occur in a matter of a few hours to days, and further represent brain changes of a different character, such as axoplasmic transport of iron or chemical processes such as the transition of iron atoms from the ferric (Fe⁺⁺⁺) to the ferrous (Fe⁺⁺) state that modifies the magnetic properties of the iron atoms.

Embodiments of the invention may also be developed and validated using noninvasive objective biomarkers for psychiatric disorders such as MDD (major depressive disorder). Also, other MRI imaging protocols, such as diffusion-weighted MRI, structural (volumetric) MRI, functional MRI (fMRI) and other types of MRI acquisitions could be adapted to provide such biomarkers in combination with the above methodologies to identify and quantify characteristics of the habenulae in relationship with various psychiatric disorders. Other imaging techniques, CT and PET in particular, may be modified or adapted to provide information on brain disorders such as depression. The implemented attributes and techniques of embodiments of the present invention would enhance the sensitivity (i.e. as in CT) and improve the spatial resolution (i.e. as in PET) without ionizing radiation to provide clinically useful information on the habenulae.

While the invention has been described in considerable detail with reference to a few exemplary embodiments only, it will be appreciated that it is not intended to limit the invention to these embodiments only, since various modifications, omissions, additions and substitutions may be made to the disclosed embodiments without materially departing from the scope of the invention. In addition, many modifications may be made to adapt to a particular situation or an installation, without departing from the essential scope of the invention. Thus, it must be understood that the above invention has been described by way of illustration and not limitation. Accordingly, it is intended to cover all modifications, omissions, additions, substitutions or the like, which may be comprised within the scope and the spirit of the invention as defined by the claims.

1. A method for diagnosing disease and monitoring therapeutic response, the method comprising the steps of:
identifying at least one neuroanatomical structure in a computed magnetic resonance image, wherein said at least one neuroanatomical structure has at least one biomarker;
generating a magnetic susceptibility map of said at least one neuroanatomical structure based on said computed magnetic resonance image; and
quantifying said biomarker associated with said at least one neuroanatomical structure to determine a diagnosis.

2. The method of claim 1, further comprising a step of monitoring said biomarker over time to diagnose a neurological condition or monitor response to a treatment plan.

3. The method of claim 1, wherein said step of monitoring said biomarker over time quantifies composition of brain matter or a therapeutic agent.

4. The method of claim 1, further comprising a step of monitoring said biomarker to determine a therapeutic response, wherein said at least one neuroanatomical structure is one or more habenula.

5. The method of claim 4, further comprising a step of utilizing a therapy targeted to said habenulae and monitoring said therapy.

6. The method of claim 2, wherein the neurological condition is diagnosed as depression.

7. The method of claim 6, wherein the habenulae are associated with depression.

8. The method of claim 2, further including a step of adjusting the treatment plan based on one or more quantitative characteristics of said biomarker.

9. The method of 1, wherein the step of generating a magnetic susceptibility map, iron concentration is measured in brain tissue.

10. The method of claim 1, further comprising a step of repeating the step of generating a magnetic susceptibility map and the step of quantifying said biomarker.

11. The method of claim 10, wherein the step of repeating occurs within hours or days to provide rapid diagnosis.

12. The method of claim 11, wherein the rapid diagnosis comprises diagnosing disease, planning treatment, modifying treatment, and studying diseases in regions of a mammalian brain.

13. The method of claim 1, further comprising a step of seeding a fiber tract map in a diffusion dataset to identify nerve bundles attached to at least one habenula.

14. The method of claim 13, wherein the step of seeding, a seed is provided to be utilized with a diffuson tensor image (DTI) to specify the nerve bundles.
